(19) United States
(12) Patent Application Publication
(10) Pub. No.: US 2019/0192110 A1
(43) Pub. Date: Jun. 27, 2019

(54) CLASSIFIER ENSEMBLE FOR DETECTION OF ABNORMAL HEART SOUNDS

(71) Applicant: KONINKLIJKE PHILIPS N.V., EINDHOVEN (NL)

(72) Inventors: Saman Parvaneh, Malden, MA (US); Cristhian Mauricio Potes Blandon, Salem, NH (US); Asif Rahman, Brookline, MA (US); Bryan Conroy, Garden City South, NY (US)

(21) Appl. No.: 16/330,252

(22) PCT Filed: Sep. 7, 2017
(86) PCT No.: PCT/EP2017/072456

§ 371 Date: Mar. 4, 2019

Related U.S. Application Data
(60) Provisional application No. 62/384,276, filed on Sep. 7, 2016.

(51) Int. Cl. A61B 7/04; A61B 5/00; G06N 3/08; G06N 3/04; G16H 50/20

(52) U.S. Cl. CPC A61B 7/04; A61B 5/7267; A61B 5/7282; G16H 50/20; A61B 5/7278; G06N 3/08; G06N 3/04; A61B 5/725

(57) ABSTRACT

Various embodiments of the inventions of the present disclosure provide a combination of feature-based approach and deep learning approach for distinguishing between normal heart sounds and abnormal heart sounds. A feature-based classifier (60) is applied to a phonocardiogram (PCG) signal to obtain a feature-based abnormality classification of the heart sounds represented by the PCG signal and a deep learning classifier (70) is also applied to the PCG signal to obtain a deep learning abnormality classification of the heart sounds represented by the PCG signal. A final decision analyzer (80) is applied to the feature-based abnormality classification and the deep learning abnormality classification of the heart sounds represented by the PCG signal to determine a final abnormality classification decision of the PCG signal.

### TECHNICAL FIELD

Various embodiments described in the present disclosure relate to systems, devices and methods for the detection of abnormal heart sounds.

### BACKGROUND

Cardiovascular diseases (CVD) are the leading cause of morbidity and mortality worldwide with an estimated 17.5 million people dying from CVD in 2012. Heart auscultation is a primary tool for screening and diagnosis CVD in primary health care. Availability of digital stethoscopes and mobile devices provides clinicians an opportunity to record and analyze heart sounds (Phonocardiogram-PCG) for diagnostic purposes.

However, an analysis of heart sounds recording in clinical and non-clinical environments has proven to be challenging due to environmental noise (e.g. alarms, speaking). Furthermore, recording heart sound by a non-expert also adds to the challenges for automatic heart sound analysis. For example, changing microphone location can change heart sound amplitude and may make it prone to noise. Additionally, the quality of heart sound recordings may be different when recorded by different equipment (e.g., due to differences in filter specification of different equipment) make the use of a single algorithm challenging. Because of above mentioned factors, feature-based approach (traditional heart sound analysis) may carry a relatively low accuracy in classification of abnormality heart sounds.

### SUMMARY

Embodiments described in the present disclosure provide a combination of feature-based approach and deep learning approach (e.g., unsupervised feature learning). More particularly, deep learning has the power to learn features from phonocardiograms designated as normal heart sounds and as abnormal heart sounds and use such abnormality features for classification purpose. The present disclosure combines benefits of feature-based classification of normal heart sounds and abnormal heart sounds and of deep learning classification of normal heart sounds and abnormal heart sounds. The present disclosure further provides for feature-based classification of noisy phonocardiogram (PCG) signals and clean PCG signals and/or of deep learning classification of noisy PCG signals and clean PCG signals.

One embodiment of the inventions of the present disclosure is a phonocardiogram (PCG) signal coanalyzer for distinguishing between normal heart sounds and abnormal heart sounds. The PCG signal coanalyzer comprises a processor and a memory configured to (1) apply a feature-based classifier to the PCG signal to obtain a feature-based abnormality classification of the heart sounds represented by the PCG signal, (2) apply a deep learning classifier to the PCG signal to obtain a deep learning abnormality classification of the heart sounds represented by the PCG signal, (3a) apply a final decision coanalyzer to the feature-based abnormality classification and the deep learning abnormality classification of the heart sounds represented by the PCG signal to determine a final abnormality classification decision of the PCG signal as normal heart sounds or abnormal heart sounds, and (4) report the final abnormality classification decision of the PCG signal.

A second embodiment of the present disclosure is the processor and the memory of the PCG signal coanalyzer being further configured to (5) apply the feature-based classifier to the PCG signal to obtain a feature-based noisy classification of the heart sounds represented by the PCG signal and (3b) apply the final decision coanalyzer to the feature-based abnormality classification, the feature-based noisy classification and the deep learning abnormality classification of the heart sounds represented by the PCG signal to determine the final abnormality classification decision of the PCG signal as normal heart sounds, abnormal heart sounds or noisy heart sounds (i.e., unsure of whether the heart sounds are normal or abnormal).

A third embodiment of the present disclosure is the processor and the memory of the PCG signal coanalyzer being further configured to (6) apply the deep learning classifier to the PCG signal to obtain a deep learning noisy classification of the heart sounds represented by the PCG signal and (3c) apply the final decision coanalyzer to the feature-based abnormality classification, the deep learning abnormality classification and the deep learning noisy classification of the heart sounds represented by the PCG signal to determine the final abnormality classification decision of the PCG signal as normal heart sounds, abnormal heart sounds or noisy heart sounds i.e., unsure of whether the heart sounds are normal or abnormal.

A fourth embodiment of the invention of the present disclosure is a non-transitory machine-readable storage medium encoded with instructions for execution by a processor for distinguishing between normal heart sounds and abnormal heart sounds, the non-transitory machine-readable storage medium comprising instructions to (1) apply a feature-based classifier to the PCG signal to obtain a feature-based abnormality classification of the heart sounds represented by the PCG signal, (2) apply a deep learning classifier to the PCG signal to obtain a deep learning abnormality classification of the heart sounds represented by the PCG signal, (3a) apply a final decision coanalyzer to the feature-based abnormality classification and the deep learning abnormality classification of the heart sounds represented by the PCG signal to determine a final abnormality classification decision of the PCG signal as normal heart sounds or abnormal heart sounds, and (4) report the final abnormality classification decision of the PCG signal.

A fifth embodiment of the present disclosure is the non-transitory machine-readable storage medium further comprising instructions to (5) apply the feature-based classifier to the PCG signal to obtain a feature-based noisy classification of the heart sounds represented by the PCG signal and (3b) apply the final decision coanalyzer to the feature-based abnormality classification, the feature-based noisy classification and the deep learning abnormality classification of the heart sounds represented by the PCG signal to determine the final abnormality classification decision of the PCG signal as normal heart sounds, abnormal heart sounds or noisy heart sounds i.e., unsure of whether the heart sounds are normal or abnormal.

A sixth embodiment of the present disclosure is the non-transitory machine-readable storage medium further comprising instructions to (6) apply the deep learning classifier to the PCG signal to obtain a deep learning noisy classification of the heart sounds represented by the PCG signal and (3c) apply the final decision coanalyzer to the feature-based abnormality classification, the deep learning abnormality classification and the deep learning noisy classification of the heart sounds represented by the PCG signal to determine the final abnormality classification decision of the PCG signal as normal heart sounds, abnormal heart sounds or noisy heart sounds i.e., unsure of whether the heart sounds are normal or abnormal.

A seventh embodiment of the inventions of the present disclosure phonocardiogram (PCG) signal coanalysis method for distinguishing between normal heart sounds and abnormal heart sounds. The PCG signal analysis method comprises (1) applying a feature-based classifier to the PCG signal to obtain a feature-based abnormality classification of the heart sounds represented by the PCG signal, (2) applying a deep learning classifier to the PCG signal to obtain a deep learning abnormality classification of the heart sounds represented by the PCG signal, (3a) applying a final decision coanalyzer to the feature-based abnormality classification and the deep learning abnormality classification of the heart sounds represented by the PCG signal to determine a final abnormality classification decision of the PCG signal as normal heart sounds or abnormal heart sounds, and (4) reporting the final abnormality classification decision of the PCG signal.

An eighth embodiment of the present disclosure is the PCG signal coanalysis method further comprising (5) applying the feature-based classifier to the PCG signal to obtain a feature-based noisy classification of the heart sounds represented by the PCG signal and (3b) applying the final decision coanalyzer to the feature-based abnormality classification, the feature-based noisy classification and the deep learning abnormality classification of the heart sounds represented by the PCG signal to determine the final abnormality classification decision of the PCG signal as normal heart sounds, abnormal heart sounds or noisy heart sounds i.e., unsure of whether the heart sounds are normal or abnormal.

A ninth embodiment of the present disclosure is the PCG signal coanalysis method further comprising (6) applying the deep learning classifier to the PCG signal to obtain a deep learning noisy classification of the heart sounds represented by the PCG signal and (3c) applying the final decision coanalyzer to the feature-based abnormality classification, the deep learning abnormality classification and the deep learning noisy classification of the heart sounds represented by the PCG signal to determine the final abnormality classification decision of the PCG signal as normal heart sounds, abnormal heart sounds or noisy heart sounds i.e., unsure of whether the heart sounds are normal or abnormal.

A tenth embodiment of the inventions of the present disclosure is a phonocardiogram (PCG) signal coanalyzer for distinguishing noisy PCG signals and clean PCG signals. The PCG signal coanalyzer comprises a processor and a memory configured to (1) apply a feature-based classifier to the PCG signal to obtain a feature-based noisy classification of the heart sounds represented by the PCG signal, (2) apply a deep learning classifier to the PCG signal to obtain a deep learning noisy classification of the heart sounds represented by the PCG signal, (3) apply a final decision coanalyzer to the feature-based noisy classification and the deep learning noisy classification of the heart sounds represented by the PCG signal to determine a final noisy classification decision of the PCG signal as a noisy PCG signal or a clean PCG signal, and (4) report the final noisy classification decision of the PCG signal.

An eleventh embodiment of the inventions of the present disclosure is a non-transitory machine-readable storage medium encoded with instructions for execution by a processor for distinguishing noisy PCG signals and clean PCG signals, the non-transitory machine-readable storage medium comprising instructions to (1) apply a feature-based classifier to the PCG signal to obtain a feature-based noisy classification of the heart sounds represented by the PCG signal, (2) apply a deep learning classifier to the PCG signal to obtain a deep learning noisy classification of the heart sounds represented by the PCG signal, (3) apply a final decision coanalyzer to the feature-based noisy classification and the deep learning noisy classification of the heart sounds represented by the PCG signal to determine a final noisy classification decision of the PCG signal as a noisy PCG signal or a clean PCG signal, and (4) report the final noisy classification decision of the PCG signal.

A twelfth embodiment of the inventions of the present disclosure is a phonocardiogram (PCG) signal coanalysis method for distinguishing between noisy PCG signals and clean PCG signals. The PCG signal analysis method comprises (1) applying a feature-based classifier to the PCG signal to obtain a feature-based noisy classification of the heart sounds represented by the PCG signal, (2) applying a deep learning classifier to the PCG signal to obtain a deep learning noisy classification of the heart sounds represented by the PCG signal, (3) applying a final decision coanalyzer to the feature-based noisy classification and the deep learning noisy classification of the heart sounds represented by the PCG signal to determine a final noisy classification decision of the PCG signal as a noisy PCG signal or a clean PCG signal, and (4) reporting the final noisy classification decision of the PCG signal.

For purposes of describing and claiming the inventions of the present disclosure:

(1) the terms “phonocardiogram”, “recorder”, “abnormality”, “normality”, “noisy”, “clean”, “feature-based”, “deep learning”, “classifier”, “classification”, “threshold”, “score” and “logical rules” are to be broadly interpreted as known in the art of the present disclosure and exemplary described in the present disclosure;

(2) the terms “coanalyze” and “coanalysis” broadly encompasses a combination of feature-based approach and a deep learning approach (e.g., unsupervised feature learning) for analyzing a PCG signal as exemplary described in the present disclosure;

(3) the term “coanalyzer” broadly encompasses a PCG analyzer as known in the art of the present disclosure or hereinafter conceived incorporating the inventive principle of present disclosure for coanalyzing a PCG signal;

(4) the terms “signal” and “data” broadly encompasses all forms of a detectable physical quantity or impulse (e.g., voltage, current, magnetic field strength, impedance, color) as understood in the art of the present disclosure and as exemplary described in the present disclosure for transmitting information and/or instructions in support of applying various inventive principles of the present disclosure as subsequently described in the present disclosure. Signal/data communication encompassed by the inventions of the present disclosure may involve any communication method as known in the art of the present disclosure including, but not limited to, data transmission/reception over any type of wired or wireless datalink and a reading of data uploaded to a computer-usable/computer readable storage medium;

(5) the descriptive labels for terms “signal” and “data” herein facilitates a distinction between signals and data as described and claimed herein without specifying or implying any additional limitation to the terms “signal” and “data”;

(6) the term “controller” broadly encompasses all structural configurations, as understood in the art of the present disclosure and as exemplary described in the present disclosure, of an application specific main board or an application specific integrated circuit for controlling an application of various inventive principles of the present disclosure as subsequently described in the present disclosure. The structural configuration of the controller may include, but is not limited to, processor(s), computer-usable/computer readable storage medium(s), an operating system, application module(s), peripheral device controller(s), slot(s) and port(s);

(7) the term “module” broadly encompasses a module incorporated within or accessible by a controller consisting of an electronic circuit and/or an executable program (e.g., executable software stored on non-transitory computer readable medium(s) and/or firmware) for executing a specific application; and

(8) the descriptive labels for term “module” herein facilitates a distinction between modules as described and claimed herein without specifying or implying any additional limitation to the term “module”.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

In order to better understand various example embodiments, reference is made to the accompanying drawings, wherein:

FIG. 1A illustrates a first exemplary embodiment of a phonocardiogram (PCG) classifier ensemble system in accordance with the present disclosure;

FIG. 1B illustrates a second exemplary embodiment of a phonocardiogram (PCG) classifier ensemble system in accordance with the present disclosure;

FIG. 2A-2J illustrates various exemplary communication modes between a PCG signal recorder and a PCG signal coanalyzer in accordance with the present disclosure;

FIG. 3 illustrates an exemplary embodiment of a PCG signal coanalysis controller in accordance with the present disclosure;

FIG. 4A illustrates an exemplary embodiment of a PCG signal conditioner in accordance with the present disclosure;

FIG. 4B illustrates an exemplary embodiment of a feature-based classifier in accordance with the present disclosure;

FIG. 4C illustrates an exemplary embodiment of a deep learning classifier in accordance with the present disclosure;

FIG. 4D illustrates an exemplary embodiment of a final decision coanalyzer in accordance with the present disclosure;

FIG. 5 illustrates an exemplary embodiment of a convolutional neural network in accordance with the present disclosure;

FIGS. 6A-6D illustrate an exemplary training of a PCG signal coanalyzer based on a set of abnormal (ab) PCG signals in accordance with the present disclosure;

FIGS. 7A-7D illustrate an exemplary training of a PCG signal coanalyzer based on a set of normal (nl) PCG signals in accordance with the present disclosure;

FIGS. 8A-8D illustrate an exemplary training of a PCG signal coanalyzer based on a set of noisy (ny) PCG signals in accordance with the present disclosure; and

FIGS. 9A-9D illustrate an exemplary training of a PCG signal coanalyzer based on a set of clean (cl) PCG signals in accordance with the present disclosure.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described in the present disclosure are not necessarily mutually exclusive and may be combined to produce additional embodiments that incorporate the principles described in the present disclosure.

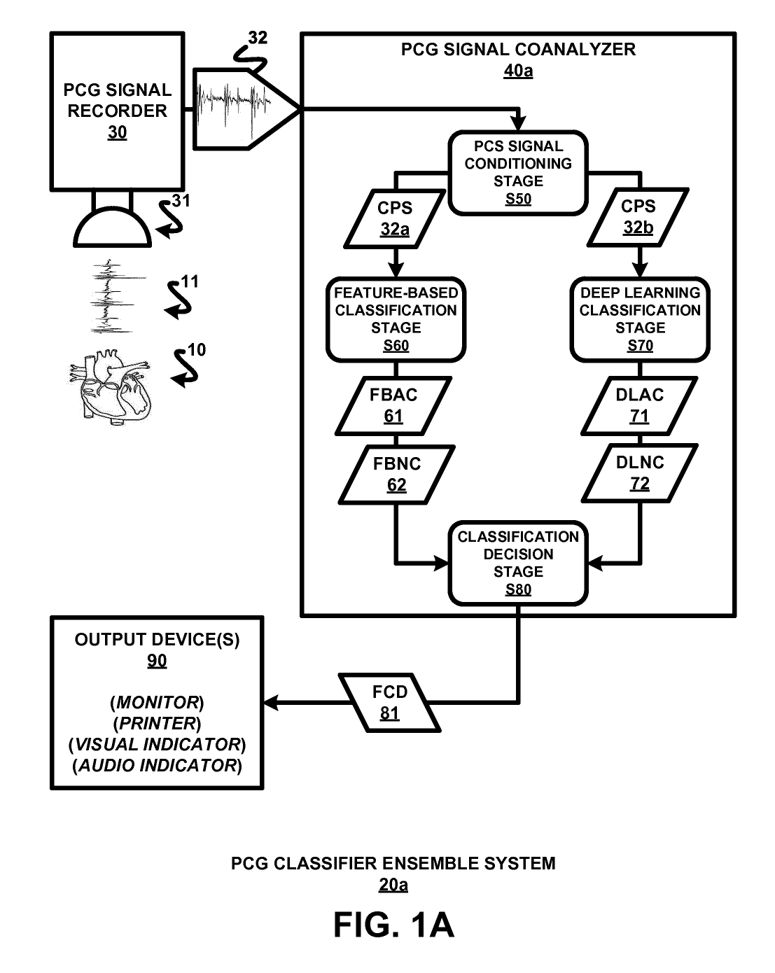

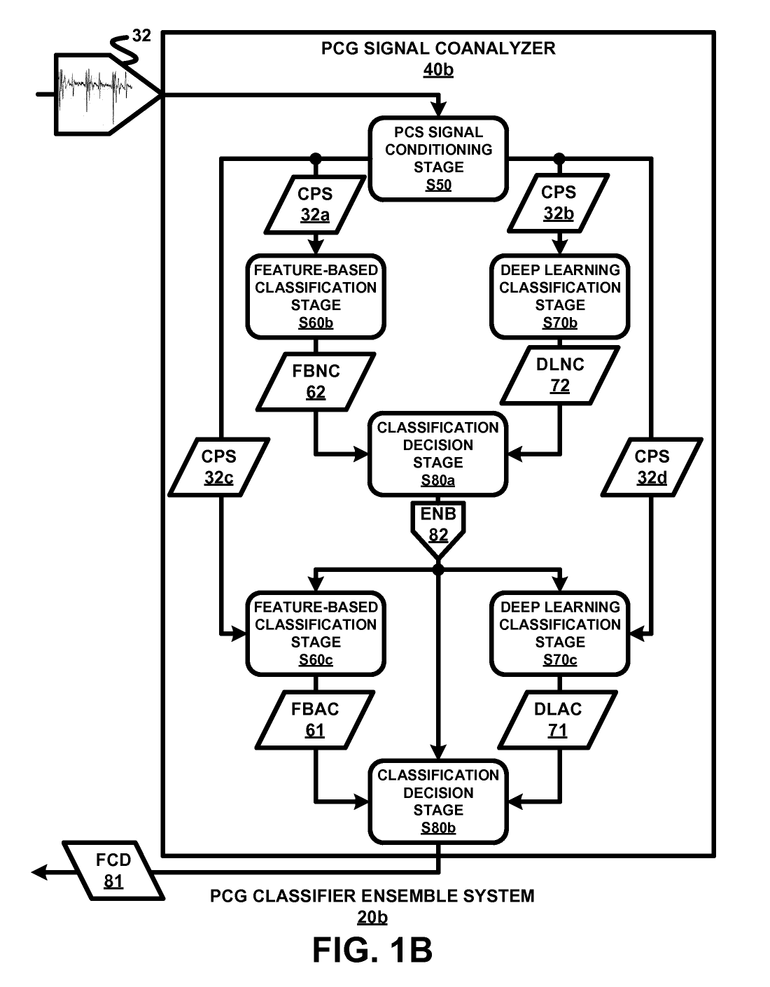

To facilitate an understanding of the inventions of the present disclosure, the following description of FIGS. 1A and 1B teaches two (2) embodiments of a PCG classifier ensemble system of the present disclosure. From the description of FIGS. 1A and 1B, those having ordinary skill in the art of the present disclosure will appreciate how to apply the present disclosure for making and using numerous and various additional embodiments of a PCG classifier ensemble system.

Referring to FIG. 1A, a PCG classifier ensemble system 20a of the present disclosure employs a PCG signal recorder 30 and a PCG signal coanalyzer 40a.

PCG signal recorder 30 is equipped with a microphone 31 to record sounds 11 of a heart 10. PCG signal recorder 30 is further configured to generate a PCG signal 32 representative of recorded sounds 11 as known in the art of the present disclosure.

PCG signal coanalyzer 40a implements a combination of a feature-based classification stage S60 and a deep learning classification stage S70 for a detection of any abnormality of hearts sounds 11 as represented by PCG signal 32 on a temporal basis or a periodic basis.

On a temporal basis involves a determination of any detection of abnormality of hearts sounds 11 as represented by PCG signal 32 for each delineated moment of time (e.g., every μsec). For example, if PCG signal 32 is being streamed from PCG signal recorder 32 to PCG signal coanalyzer 40a in real-time, then PCG signal coanalyzer 40a individually evaluates each delineated moment of time for any abnormality of hearts sounds 11 as represented by PCG signal 32.

On a periodic basis involves a determination of any detection of abnormality of hearts sounds 11 as represented by PCG signal 32 over a period of time in terms of second, minutes or hours. For example, if a pre-recorded PCG signal 32 is being uploaded to PCG signal coanalyzer 40a, then PCG signal coanalyzer 40a evaluates the pre-recorded signal 32 over a period of time for any abnormality of hearts sounds 11 as represented by PCG signal 32.

Still referring to FIG. 1A, PCG signal coanalyzer 40a optionally implements a PCG signal conditioning stage S50 involving a conditioning of PCG signal 32 as needed to prepare PCG signal 32 for classifier(s) of feature-based classification stage S60 and/or deep learning classification stage S70.

In practice, conditioning techniques applied by PCG signal conditioning stage S50 will be dependent upon a condition of PCG signal 32 as received by PCG signal coanalyzer 40a and/or upon a particular type of classifier implemented by feature-based classification stage S60 and/or a deep learning classification stage S70.

In a first embodiment of PCG signal conditioning stage S50, PCG signal 32 may be resampled and filtered as further exemplary described in the present disclosure.

In a second embodiment of PCG signal conditioning stage S50, PCG signal 32 may be segmented into numerous heart states (e.g., a heart state 51, a systole heart state, a heart state S2 and a diastole heart state) as further exemplary described in the present disclosure to thereby facilitate an application of classifier(s) of feature-based classification stage S60 and/or deep learning classification stage S70.

Also in practice, PCG signal conditioning stage S50 may apply the same conditioning techniques to PCG signal 31 resulting in a conditioned PCG signal 32a for feature-based classification stag S60 and a conditioned PCG signal 32b for deep learning classification stage S70 being identical. Alternatively, PCG signal conditioning stage S50 may apply different conditioning techniques to PCG signal 31 resulting in conditioned PCG signal 32a for deep learning classification stag S60 and a conditioned PCG signal 32b for deep learning classification stage S70 being dissimilar.

Still referring to FIG. 1A, feature-based classification stage S60 involves an application of a feature-based classifier to PCG signal 32 or a conditioned PCG signal 32a on a temporal basis or a periodic basis to thereby obtain a feature-based abnormality classification 61 of the heart sounds of PCG signal 32 or a conditioned PCG signal 32a.

In practice, feature-based classification stage S60 may implement any type of feature-based classifier configurable for providing a quantitative score of a degree of abnormality of PCG signal 32 or conditioned PCG signal 32b.

In a first embodiment of feature-based classification stage S60, a feature-based classifier is trained to create a model for deriving feature-based abnormality classification 61 from extracted features of PCG signal 32 or conditioned PCG signal 32b whereby feature-based abnormality classification 61 is a comprehensive quantitative score of a degree of abnormality of each extracted feature of PCG signal 32 or conditioned PCG signal 32b on a temporal basis or a periodic basis as will be further exemplary described in the present disclosure.

Feature-based classification stage S60 may further involve an application of a feature-based classifier to PCG signal 32 or a conditioned PCG signal 32a on a temporal basis or a periodic basis to thereby obtain a feature-based noisy classification 62 of the heart sounds of PCG signal 32 or a conditioned PCG signal 32a.

In practice, feature-based classification stage S60 may implement any type of feature-based classifier configurable for providing a quantitative score of both a degree of abnormality and a degree of noise of PCG signal 32 or conditioned PCG signal 32b.

In a second embodiment feature-based classification stage S60, the feature-based classifier is further trained to create a model for deriving feature-based noisy classification 62 from the same, different or overlapping extracted features of PCG signal 32 or conditioned PCG signal 32b whereby feature-based noisy classification 62 is a comprehensive quantitative score of a degree of noise of each extracted feature of PCG signal 32 or conditioned PCG signal 32b on a temporal basis or a periodic basis as will be further exemplary described in the present disclosure.

Still referring to FIG. 1A, deep learning classification stage S70 involves an application of a deep learning classifier to PCG signal 32 or a conditioned PCG signal 32a on a temporal basis or a periodic basis to thereby obtain a deep learning abnormality classification 71 of the heart sounds of PCG signal 32 or a conditioned PCG signal 32a.

In practice, deep learning classification stage S70 may implement any type of deep learning classifier configurable for providing a quantitative score of a degree of abnormality of PCG signal 32 or conditioned PCG signal 32b.

In a first embodiment of deep learning classification stage S70, the deep learning classifier is trained to create a model for deriving deep learning abnormality classification 71 from decomposed frequency bands of PCG signal 32 or conditioned PCG signal 32b whereby deep learning abnormality classification 71 is a comprehensive quantitative score of a degree of noise of each decomposed frequency band of PCG signal 32 or conditioned PCG signal 32b on a temporal basis or a periodic basis as will be further exemplary described in the present disclosure.

Deep learning classification stage S70 may further involve an application of the deep learning classifier to PCG signal 32 or a conditioned PCG signal 32a on a temporal basis or a periodic basis to thereby obtain a deep learning noisy classification 72 of the heart sounds of PCG signal 32 or a conditioned PCG signal 32a.

In practice, deep learning classification stage S70 may implement any type of deep learning classifier configurable for providing a quantitative score of both a degree of abnormality and a degree of noise of PCG signal 32 or conditioned PCG signal 32b.

In second embodiment of deep learning classification stage S70, the deep learning classifier is further trained to create a model for deriving deep learning noisy classification 72 from the same, different or overlapping frequency bands of PCG signal 32 or conditioned PCG signal 32b whereby deep learning noisy classification 72 is a comprehensive quantitative score of a degree of noise of each decomposed frequency bands of PCG signal 32 or conditioned PCG signal 32b on a temporal basis or a periodic basis as will be further exemplary described in the present disclosure.

Still referring to FIG. 1A, PCG signal coanalyzer 40a further implements a a classification decision stage S80 involving an application of a final decision coanalyzer to both feature-based abnormality classification 61 and deep learning abnormality classification 71 to thereby determine a final abnormality classification decision 81 indicating any detection of an abnormality of the heart sounds represented by PCG signal 32 on a temporal basis or a periodic basis.

In practice, the final decision coanalyzer may implement one or more logical rules for determining whether feature-based abnormality classification 61 and deep learning abnormality classification 71 collectively indicate any detection of an abnormality of the heart sounds represented by PCG signal 32.

In a first embodiment of classification decision stage S80, the final decision coanalyzer may determine a detection of an abnormality of the heart sounds represented by PCG signal 32 on a temporal basis or a periodic basis if both feature-based abnormality classification 61 and deep learning abnormality classification 71 indicate a detection of an unacceptable degree of abnormality of the heart sounds represented by PCG signal 32 derived from a comparison of feature-based abnormality classification 61 and deep learning abnormality classification 71 to abnormal classification threshold(s) as will be further exemplary described in the present disclosure.

Also in practice, for embodiments involving a detection of any noise in PCG signal 32, the final decision coanalyzer may implement one or more logical rules for conditionally determining whether feature-based abnormality classification 61 and deep learning abnormality classification 71 collectively indicate any detection of an abnormality of the heart sounds represented by PCG signal 32 based on the degree of noise in PCG signal 32.

In a second embodiment of classification decision stage S80, the final decision coanalyzer may conditionally determine a detection of an abnormality of the heart sounds represented by PCG signal 32 as set forth in the first embodiment of classification decision stage S80 if both feature-based noisy classification 62 and/or deep learning noisy classification 72 fail to indicate a detection of an unacceptable degree on noise within the heart sounds represented by PCG signal 32 derived from a comparison of feature-based noisy classification 62 and/or deep learning noisy classification 72 to noisy classification threshold(s) as will be further exemplary described in the present disclosure.

Still referring to FIG. 1A, classification decision stage S80 further involves a reporting of final abnormality classification decision 81 to a clinician, etc. via one or more output devices 90 including, but not limited to, a monitor (e.g., of a workstation, a mobile device), a printer, a visual indicator (e.g., an LED assembly) and an audio indicator (e.g., a speaker).

In practice, final abnormality classification decision 81 may be communicated to output device(s) 90 in any format suitable for a notification of final abnormality classification decision 81 to a clinician, etc. More particularly in practice, final abnormality classification decision 81 may simply be reported as representing normal heart sounds or abnormal heart sounds, or as a noisy PCG signal (if applicable). Furthermore, a reporting of final abnormality classification decision 81 may include additional information, such as, for example, a degree of abnormality of the heart sounds or a notification to re-do a hear sound recording via PCG signal recorder 30 for a noisy PCG signal (if applicable).

Also in practice, an output device 90 may be a component of PCG signal recorder 30 or PCG signal coanalyzer 40a.

Referring to FIG. 1B, a PCG classifier ensemble system 20b of the present disclosure employs a PCG signal recorder 30 (FIG. 1A) and a PCG signal coanalyzer 40b.

For system 20b, PCG signal coanalyzer 40b utilizes feature-based noisy classification 62 and/or deep learning noisy classification 72 as enabling signals for determining whether feature-based abnormality classification 61 and deep learning abnormality classification 71 collectively indicate any detection of an abnormality of the heart sounds represented by PCG signal 32.

Specifically, PCG signal coanalyzer 40b optionally implements PCG conditioning stage S50 as previously described in the present disclosure for generating conditioned PCG signals 32a-32d, which may be the same conditioned PCG signals, different conditioned PCG signals or a combination thereof.

PCG signal coanalyzer 40b implements a feature-based classification stage S60a for obtaining feature-based noisy classification 62 as previously described in the present disclosure and/or a deep learning classification stage S70a for obtaining deep learning noisy classification 72 as previously described in the present disclosure whereby a classification decision stage S80a generates an enablement signal 82 for enabling or disabling a feature-based classification stage S60b, a deep learning classification stage S70b and a classification decision stage S80b dependent upon a degree of noise within PCG signal 32 as indicated individually or collectively by feature-based noisy classification 62 and/or deep learning noisy classification 72. If enabled, feature-based classification stage S60b, deep learning classification stage S70b and classification decision stage S80b are implemented as previously described in the present disclosure for a reporting of final abnormality classification decision 81 to a clinician, etc. via one or more output devices 90 (FIG. 1A).

Still referring to FIG. 1B, PCG signal analyzer 40b may omit S60c, S70c and S80c whereby stage S80 alternatively outputs a final noisy classification decision of PCG signal 32 instead on enablement signal 82. The final noisy classification decision of PCG signal 32 may be reported as a noisy PCG signal or a clean PCG signal. Furthermore, a reporting of PCG signal 32 as a noisy PCG signal may include additional information, such as, for example, a notification to re-do a hear sound recording via PCG signal recorder 30.

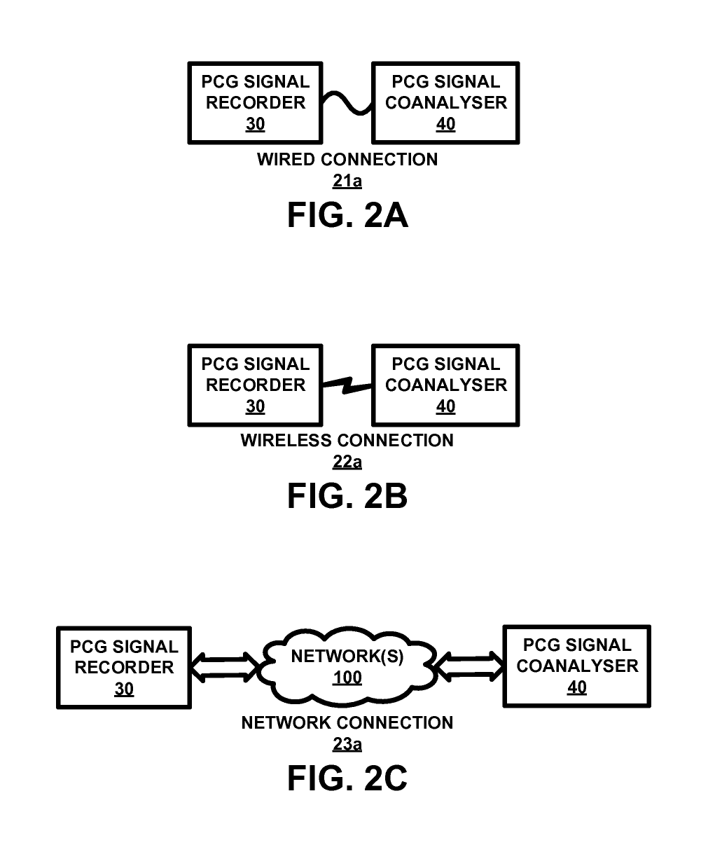

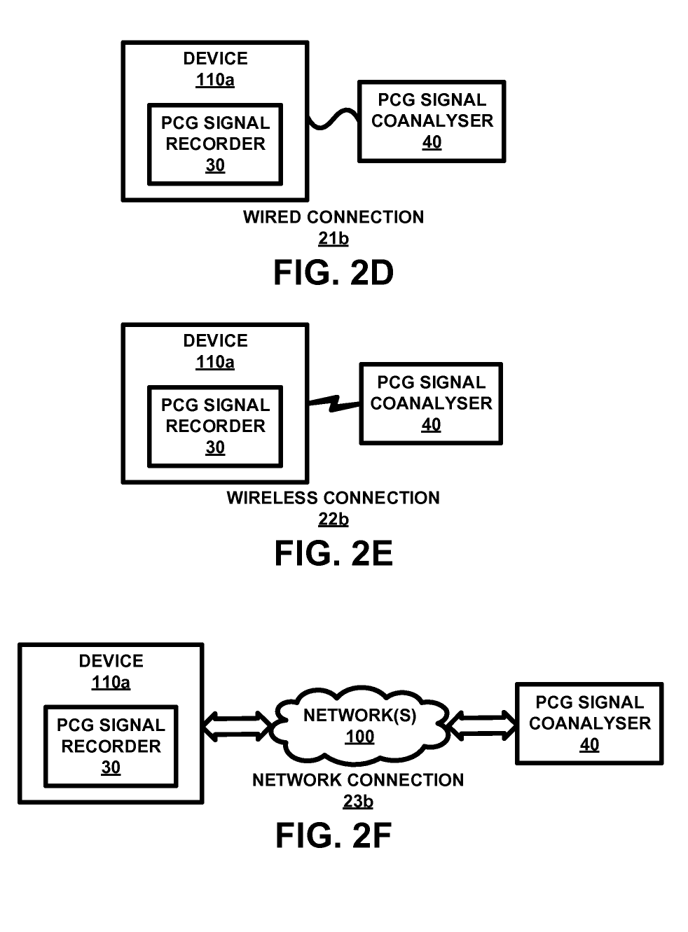

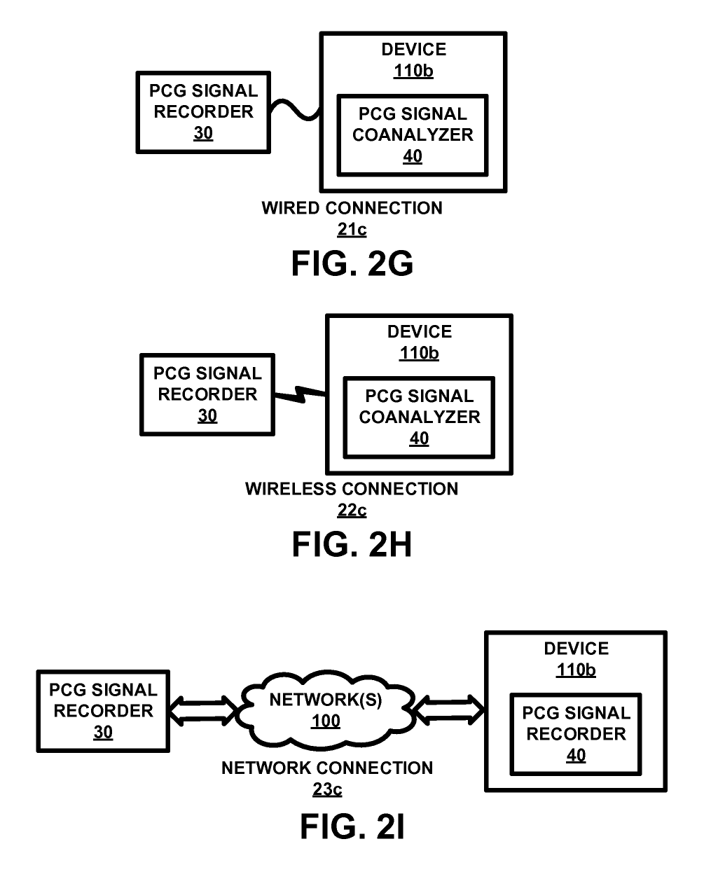

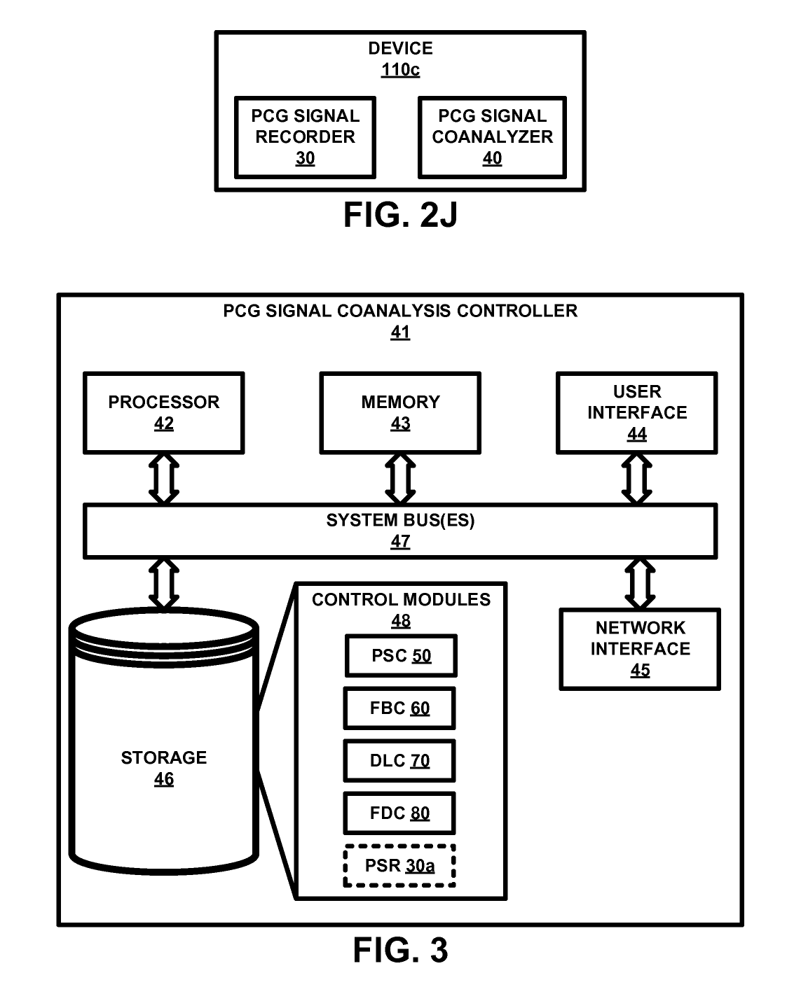

To facilitate a further understanding of the inventions of the present disclosure, the following description of FIGS. 2A-2J teaches various embodiments of communication modes between a PCG signal recorder and a PCG signal coanalyzer of the present disclosure. From the description of FIGS. 2A-2J, those having ordinary skill in the art of the present disclosure will appreciate how to apply the present disclosure for making and using numerous and various additional embodiments of communication modes between a PCG signal recorder and a PCG signal coanalyzer.

Referring to FIGS. 2A-2C, PCG signal recorder 30 (FIG. 1A) and a PCG signal coanalyzer 40 (FIGS. 1A and 1B) are shown as stand-alone devices. For example, PCG signal recorder 30 may be a digital stethoscope and PCG signal coanalyzer 40 may be a PCG monitor. FIG. 2A further shows an implementation of a wired communication 21a between PCG signal recorder 30 and PCG signal coanalyzer 40. FIG. 2B further shows an implementation of a wired communication 22a between PCG signal recorder 30 and PCG signal coanalyzer 40. FIG. 2C further shows an implementation of a wired/wireless network communication 23a between PCG signal recorder 30 and PCG signal coanalyzer 40 via one or more networks 100 of any type.

Referring to FIGS. 2D-2F, PCG signal recorder 30 is shown as a component of a device 110a and PCG signal coanalyzer 40 is shown as a stand-alone device. For example, PCG signal recorder 30 may a component of a handheld device of any type and PCG signal coanalyzer 40 may be a PCG monitor. FIG. 2D further shows an implementation of a wired communication 21b between device 110a and PCG signal coanalyzer 40. FIG. 2E further shows an implementation of a wired communication 22b between device 110a and PCG signal coanalyzer 40. FIG. 2F further shows an implementation of a wired/wireless network communication 23b between device 110a and PCG signal coanalyzer 40 via one or more networks 100 of any type.

Referring to FIGS. 2G-2I, PCG signal recorder 30 is shown as a stand-alone device and PCG signal coanalyzer 40 is shown as a component of a device 110b. For example, PCG signal recorder 30 may be a digital stethoscope and PCG signal coanalyzer 40 may a component of a handheld device. FIG. 2G further shows an implementation of a wired communication 21c between PCG signal recorder 30 and device 110b. FIG. 2H further shows an implementation of a wired communication 22c between PCG signal recorder 30 and device 110b. FIG. 2I further shows an implementation of a wired/wireless network communication 23c between PCG signal recorder 30 and device 110b via one or more networks 100 of any type.

In practice, a wired, wireless or network communication may also be implemented for device 110a (FIGS. 2D-2F) and device 110b (FIGS. 2G-2I).

Referring to FIG. 2J, PCG signal recorder 30 and PCG signal coanalyzer 40 are both shown as components of a device 110c whereby PCG signal recorder 30 and PCG signal coanalyzer 40 may be integrated or segregated components of device 110c.

To facilitate a further understanding of the inventions of the present disclosure, the following description of FIGS. 3-9C teaches various embodiments of a PCG signal coanalysis controller of the present disclosure. From the description of FIGS. 3-9C, those having ordinary skill in the art of the present disclosure will appreciate how to apply the present disclosure for making and using numerous and various additional embodiments of a PCG signal coanalysis controller

FIG. 3 illustrates a PCG signal coanalysis controller 41 for implementing stages S50-S80 of FIGS. 1A and 1B. As shown, controller 41 includes a processor 42, a memory 43, a user interface 44, a network interface 45, and a storage 46 interconnected via one or more system bus(es) 48. In practice, the actual organization of the components 42-47 of controller 41 may be more complex than illustrated.

The processor 42 may be any hardware device capable of executing instructions stored in memory or storage or otherwise processing data. As such, the processor 42 may include a microprocessor, field programmable gate array (FPGA), application-specific integrated circuit (ASIC), or other similar devices.

The memory 43 may include various memories such as, for example L1, L2, or L3 cache or system memory. As such, the memory 43 may include static random access memory (SRAM), dynamic RAM (DRAM), flash memory, read only memory (ROM), or other similar memory devices.

The user interface 44 may include one or more devices for enabling communication with a user such as an administrator. For example, the user interface 44 may include a display, a mouse, and a keyboard for receiving user commands. In some embodiments, the user interface 44 may include a command line interface or graphical user interface that may be presented to a remote terminal via the network interface 45.

The network interface 45 may include one or more devices for enabling communication with other hardware devices. For example, the network interface 45 may include a network interface card (NIC) configured to communicate according to the Ethernet protocol. Additionally, the network interface 45 may implement a TCP/IP stack for communication according to the TCP/IP protocols. Various alternative or additional hardware or configurations for the network interface will be apparent.

The storage 46 may include one or more machine-readable storage media such as read-only memory (ROM), random-access memory (RAM), magnetic disk storage media, optical storage media, flash-memory devices, or similar storage media. In various embodiments, the storage 46 may store instructions for execution by the processor 42 or data upon with the processor 42 may operate. For example, the storage 46 store a base operating system (not shown) for controlling various basic operations of the hardware.

More particular to the present disclosure, storage 46 further stores control modules 48 including a PCG signal conditioner 50 for implementing PCG signal conditioning stage S50 (FIGS. 1A and 1B), one or more feature-based classifiers 60 for implementing one or more feature-based classification stages S60 (FIGS. 1A and 1B), one or more deep learning classifiers 70 for implementing one or more deep learning classification stages S70 (FIGS. 1A and 1B), and one or more final decision coanalyzers 80 for implementing classification decision stages S80 (FIGS. 1A and 1B).

Control modules 48 may further include PCG signal recorder 30a for embodiments having an integration of a PCG signal recorder 30 and a PCG signal coanalyzer 40.

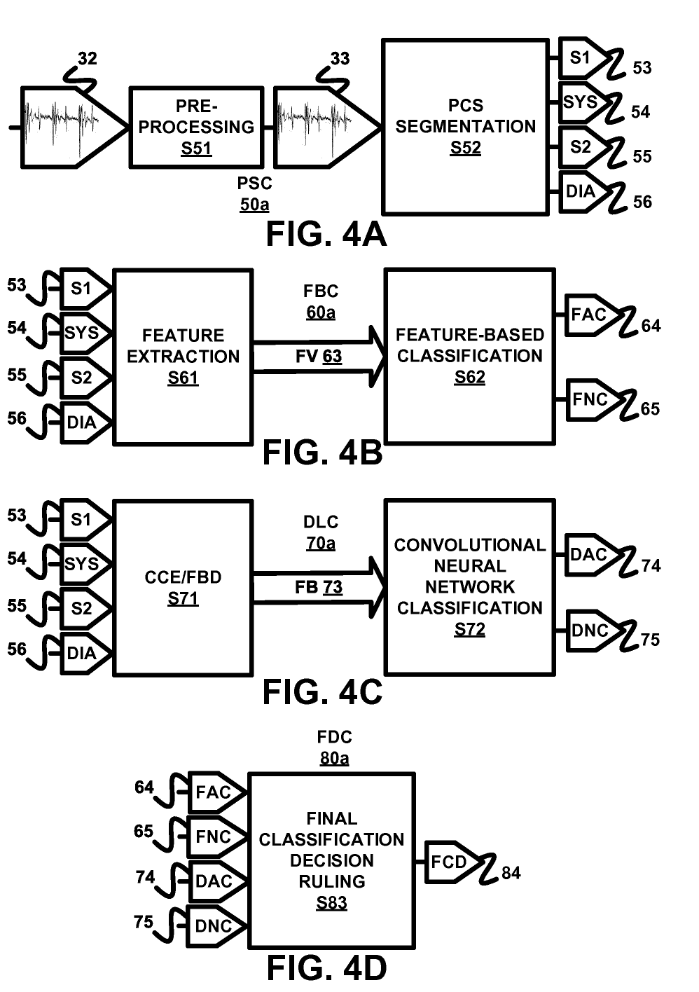

Referring to FIG. 4A, an exemplary embodiment 50a of PCG signal conditioner 50 (FIG. 3) implements a pre-processing stage S51 and a PCS signal segmentation stage S52.

Pre-processing stage S51 involves a resampling of PCG signal 32 to 1000 Hz, band-pass filtered between 25 Hz and 400 Hz, and then pre-processed to remove any spikes in PCG signal 32 as known in the art of the present disclosure.

PCS signal segmentation stage S52 involves a segmenting of a resampled/filtered PCG signal 33 into a S1 heart sound state signal 53, a systole heart sound state signal 54, a S2 heart sound state signal 55 and a diastole heart sound state signal 56 using a segmentation method as known in the art of the present disclosure (e.g., a logistic regression segmentation method).

Referring to FIG. 4B, an exemplary embodiment 60a of feature-based classifier 60 implements a feature extraction stage S61 and a feature-based classification stage S62.

Feature extraction stage S61 involves a feature vector 63 derived from an extraction of one or more time-domain features and/or one or more frequency-domain features from heard sound state signals 53-56.

In a first embodiment of feature extraction stage S61, statistical features (e.g., a mean and standard deviation (SD)) of PCG interval parameters and PCG amplitude parameters were used as thirty-six (36) time-domain features.

The PCG interval parameters may include RR intervals, S1 intervals, S2 intervals, systolic intervals, diastolic intervals, ratio of systolic interval to RR interval of each heartbeat, ratio of diastolic interval to RR interval of each heartbeat, and/or ratio of systolic to diastolic interval of each heartbeat.

The PCG amplitude parameters may include ratio of the mean absolute amplitude during systole to that during the S1 period in each heartbeat, ratio of the mean absolute amplitude during diastole to that during the S2 period in each heartbeat, skewness of the amplitude during S1 period in each heartbeat, skewness of the amplitude during S2 period in each heartbeat, skewness of the amplitude during systole period in each heartbeat, skewness of the amplitude during diastole period in each heartbeat, kurtosis of the amplitude during S1 period in each heartbeat, kurtosis of the amplitude during S2 period in each heartbeat, kurtosis of the amplitude during systole period in each heartbeat, and/or kurtosis of the amplitude during diastole period in each heartbeat.

In a second embodiment of feature extraction stage S61, for each cardiac cycle, a time series for each heart sound state signal 53-56 is created for frequency analysis. A frequency spectrum is estimated using a Hamming window and a discrete-time Fourier transform. The median power across nine (9) frequency bands (e.g., 25-45 Hz, 45-65 Hz, 65-85 Hz, 85-105 Hz, 105-125 Hz, 125-150 Hz, 150-200 Hz, 200-300 Hz and 300-400 Hz) in time series with S1, S2, systole, and diastole for each cardiac cycle is calculated. T hen, mean of median power in different bands for all cycles are used as thirty-six (26) frequency-domain features.

Additionally, thirteen (13) mel-frequency cepstral coefficient (MFCC) may be extracted from heard sound state signals 53-56 in each cardiac cycle and a mean of MFCCs for different cycles for different heart sound states may be used as fifty-two (52) MFCC features.

Feature-based classification S62 involves an implementation of a AdaBoost-abstain classifier. Specifically, AdaBoost is an effective machine learning technique for building a powerful classifier from an ensemble of “weak learners”, whereby the boosted classifier H(x) is modeled as a generalized additive model of many base hypotheses in accordance with the following equation [1]:

$$H ( x ) = b + {\sum }_{t} {α}_{t} h ( x ; {θ}_{t} ) \qquad [ 1 ]$$

where b is a constant bias that accounts for the prevalence of the categories, and where each base classifier h(x; θₜ) is a function of x, with parameters given by the elements in the vector θₜ, and produces a classification output (+1 or −1).

In practice, each base classifier is a simple decision stump over one of the above features whereby each base classifier is configured to abstain from voting (output=0) using a modified version AdaBoost-abstain of AdaBoost. A final classification decision is assigned by taking the sign of H(x), which results in a weighted majority vote over the base classifiers in the model.

In a first embodiment of feature-based classification S62, a preliminary classification decision is a feature-based abnormality decision 64 specifying a quantitative score of a degree of abnormality of the heart sounds represented by PCG signal 32.

In a second embodiment of feature-based classification S62, the preliminary classification decision additional includes a feature-based noisy decision 65 specifying a quantitative score of a degree of noise within PCG signal 32.

Referring to FIG. 4C, an exemplary embodiment 70a of deep learning classifier 70 implements a cardiac cycles extraction/frequency bands decomposition stage S71 and a convolutional neural network (CNN) classification stage S72.

Cardiac cycles extraction/frequency bands decomposition stage S71 involves extracting cardiac cycles from heart sound state signals 53-56 and decomposing of each cardiac cycle into four (4) frequency bands 73 (i.e. 25-45 Hz, 45-80 Hz, 80-200 Hz, and 200-400 Hz). Each cardiac cycle had a fixed duration (e.g., 2.5 seconds) corresponding to an anticipated longest cardiac cycle of PCG signal 32. If a cardiac cycle of PCG signal 32 has a shorter duration, then the time series is zero padded.

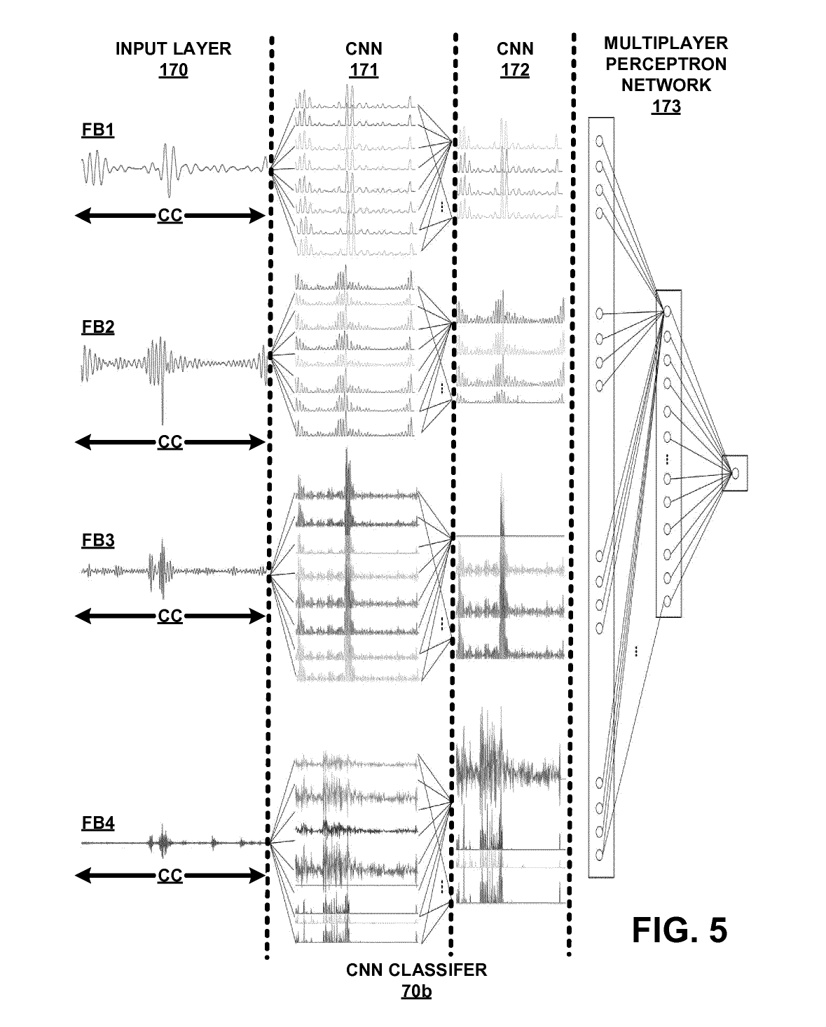

CNN classification stage S72 involves a processing of frequency bands 73 by a CNN classifier 70b shown in FIG. 5.

Referring to FIG. 5, four (4) time series, one per each frequency band, are the inputs to CNN classifier 70b. Each of CNN classifiers 70b consist of three layers, an input layer 170 followed by two (2) convolution layer 171 and 172. Input layer 170 corresponds to the cardiac cycle of a specific frequency band (i.e. length=2500 samples). Each convolutional layer 171 and 172 involves a convolution operation, a nonlinear transformation, and a maxpooling operation. The first convolutional layer 171 has eight (8) filters of length 5, followed by ReLu, and a max-pooling of 2. The second convolutional layer 172 has four (4) filters of length 5, followed by ReLu, and a max-pooling of 2. The outputs of convolutional layer 172 are inputted to a multilayer perceptron (MLP) network 173, which consists of an input layer (i.e., a flattened output of CNN 172, a hidden layer with twenty (20) neurons, and an output layer (i.e. one node). The activation function in the hidden layer of network 173 is a ReLu and the activation function in the output layer of network 173 is a sigmoid. The output layer of network 172 computes the class score (e.g., a probability value, CNN_ABN) of abnormal heart sound. Dropout of 25% may be applied after max-pooling of the second convolutional layer 172. Dropout of 50% and L2 regularization may be applied at the hidden layer of the MLP network 173.

In a first embodiment of CNN classification stage S72, a preliminary classification decision is a deep learning abnormality decision 74 specifying a quantitative score of a degree of abnormality of the heart sounds represented by PCG signal 32.

In a second embodiment of CNN classification stage S72, the preliminary classification decision additional includes a deep learning noisy decision 75 specifying a quantitative score of a degree of noise within PCG signal 32.

Referring to FIG. 4D, an exemplary embodiment 80a of final decision coanalyzer 80 implements a final classification ruling stage S83 involving a coanalysis of the preliminary classification decisions to determine a final abnormality classification decision 84 of the heart sounds represented by PCG signal 32.

In a first embodiment of final classification ruling stage S83, the following logical decision rule is applied to feature-based abnormality classification 64 (AdaBoost_ABN) and deep learning abnormality classification 74 (CNN_ABN) based on a feature-based abnormality threshold (thr_ABN) and a deep learning abnormality threshold (thr_CNN):

Algorithm 1 Decision Rule

if (Adaboost_ABN) > thr_ABN) V (CNN_ABN > thr_CNN) then

Abnormal PCG

else

Normal PCG

end if

In a second embodiment of final classification ruling stage S83, the following logical decision rule is applied to feature-based abnormality classification 64 (AdaBoost_ABN), deep learning abnormality classification 74 (CNN_ABN) and feature-based noisy classification 65 (AdaBoost_SQI) based on a feature-based abnormality threshold (thr_ABN), a deep learning abnormality threshold (thr_CNN) and a feature-based noisy threshold (thr_SQI):

Algorithm 2 Decision Rule

if Adaboost_SQI > thr_SQI then

Unsure PCG

else
if (Adaboost_ABN) > thr_ABN) V (CNN_ABN > thr_CNN) then

Abnormal PCG

else

Normal PCG

end if
end if

In a third embodiment of final classification ruling stage S83, the following logical decision rule is applied to feature-based abnormality classification 64 (AdaBoost_ABN), deep learning abnormality classification 74 (CNN_ABN) and deep learning noisy classification 75 (CNN_SQI) based on a feature-based abnormality threshold (thr_ABN), a deep learning abnormality threshold (thr_CNN) and a deep learning noisy threshold (thr_SQI):

Algorithm 2 Decision Rule

if CNN_SQI > thr_SQI then

Unsure PCG

else
if (Adaboost_ABN) > thr_ABN) V (CNN_ABN > thr_CNN) then

Abnormal PCG

else

Normal PCG

end if
end if

In a fourth embodiment of final classification ruling stage S83, the following logical decision rule is applied to feature-based abnormality classification 64 (AdaBoost_ABN), deep learning abnormality classification 74 (CNN_ABN), feature-based noisy classification 65 (AdaBoost_SQIA) and deep learning noisy classification 75 (CNN_SQIC) based on a feature-based abnormality threshold (thr_ABN), a deep learning abnormality threshold (thr_CNN), a feature-based noisy threshold (thr_SQIA) and a deep learning noisy threshold (thr_SQIC):

Algorithm 2 Decision Rule

if (Adaboost_SQIA > thr_SQIA) ∧ (CNN_SQI > thr_SQIC) then

Unsure PCG

else
if (Adaboost_ABN) > thr_ABN) V (CNN_ABN > thr_CNN) then

Abnormal PCG

else

Normal PCG

end if
end if

For embodiments of the present disclosure for distinguishing between noisy PCG signals and clean PCG signals, a feature-based classifier of the present disclosure will generate a feature-based noisy classification as previously described in the present disclosure and a deep learning classifier of the present disclosure will generate a deep learning noisy classification whereby a final decision coanalyzer will apply logical rules to the feature-based noisy classification and the deep learning noisy classification to determine a final noisy classification decision. For example, feature-based noisy classification may be compared to a feature-based noisy threshold and a deep learning noisy classification may be compared to a deep learning noisy threshold whereby a logical AND or a logic OR is applied to the comparison results to determine the final noisy classification decision.

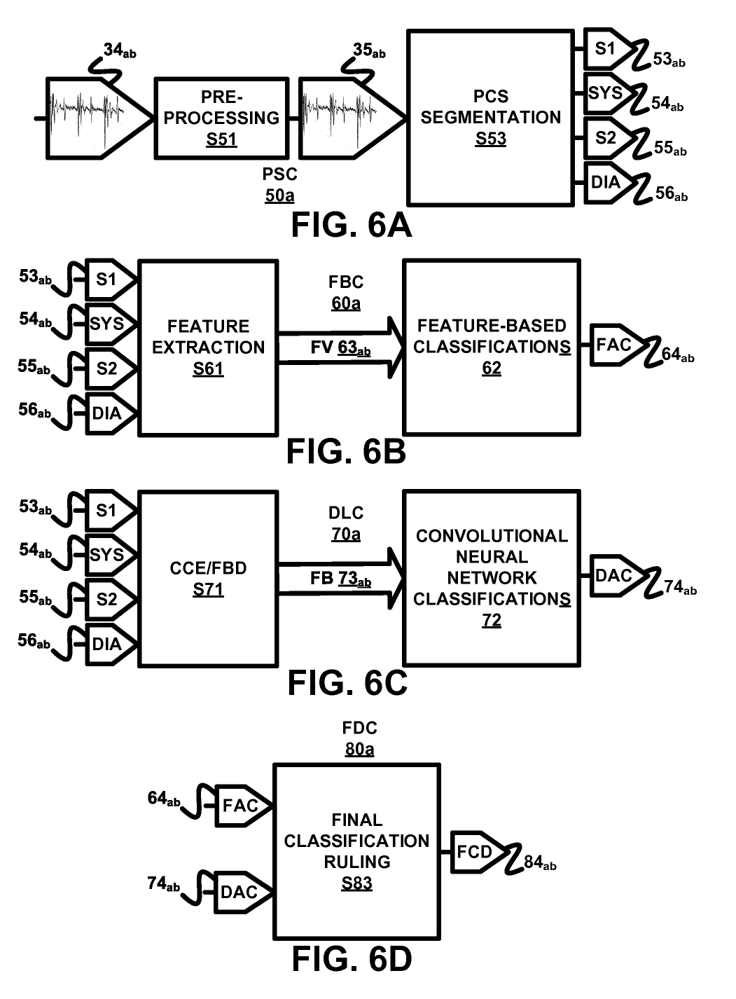

FIGS. 6A-6D illustrate an exemplary training of feature-based classifier 60a, deep learning classifier 70a and final decision coanalyzer 80a based on a training set of PCG signals 32_{ab} delineated as represented abnormal heart sounds.

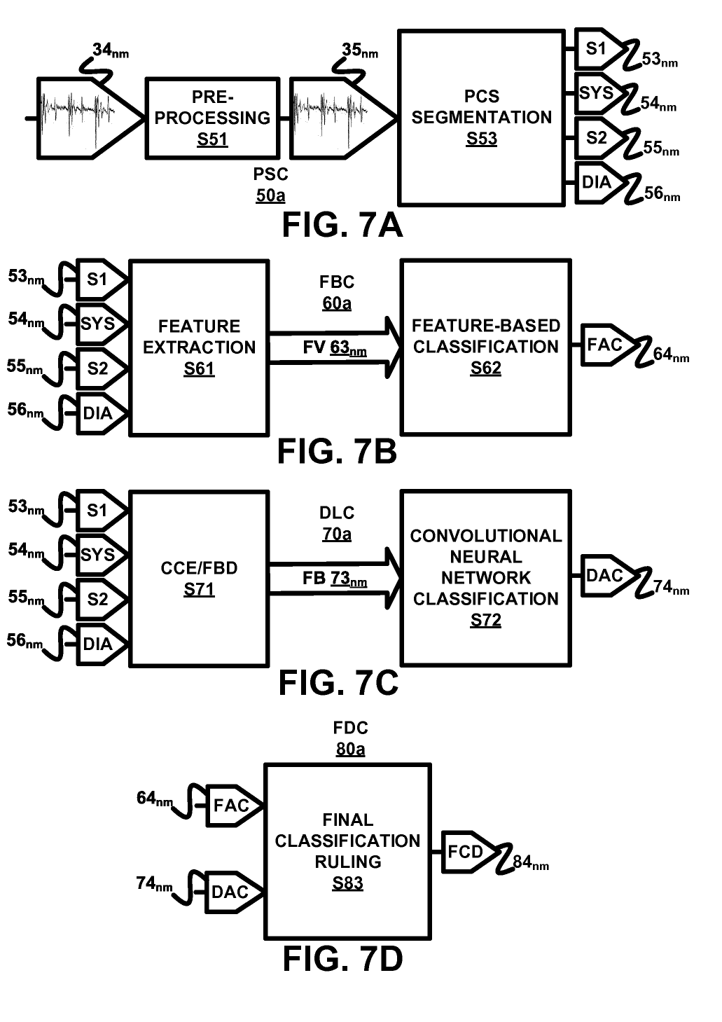

FIGS. 7A-7D illustrate an exemplary training of feature-based classifier 60a, deep learning classifier 70a and final decision coanalyzer 80a based on a training set of PCG signals 32ₙₘ delineated as represented normal heart sounds.

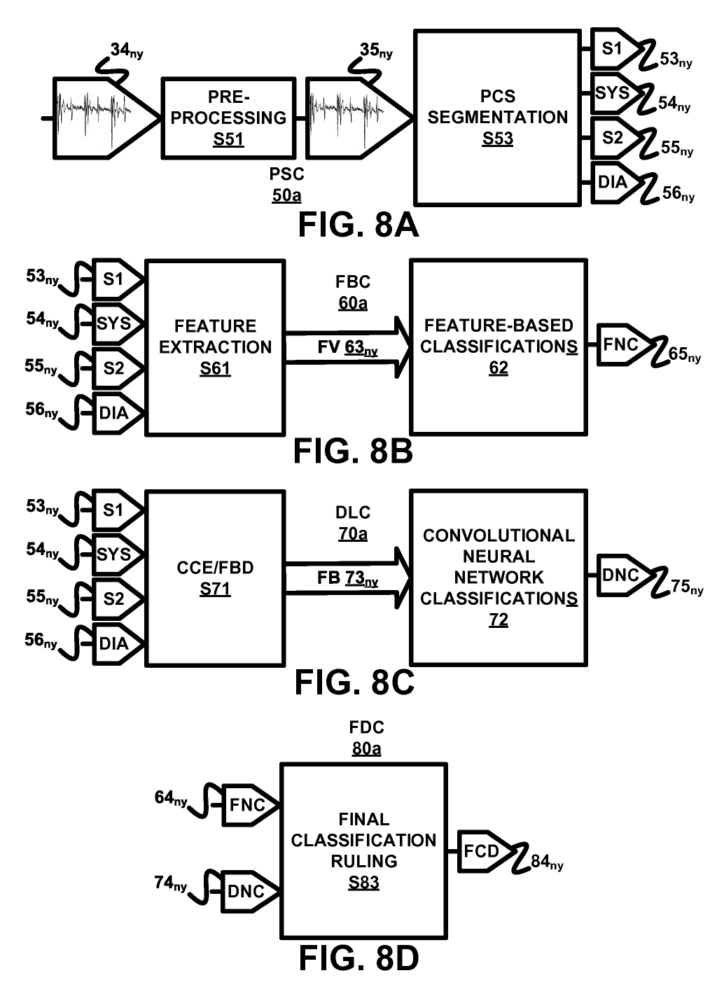

FIGS. 8A-8D illustrate an exemplary training of feature-based classifier 60a, deep learning classifier 70a and final decision coanalyzer 80a based on a training set of PCG signals 32_{ab} delineated as noisy PCG signals.

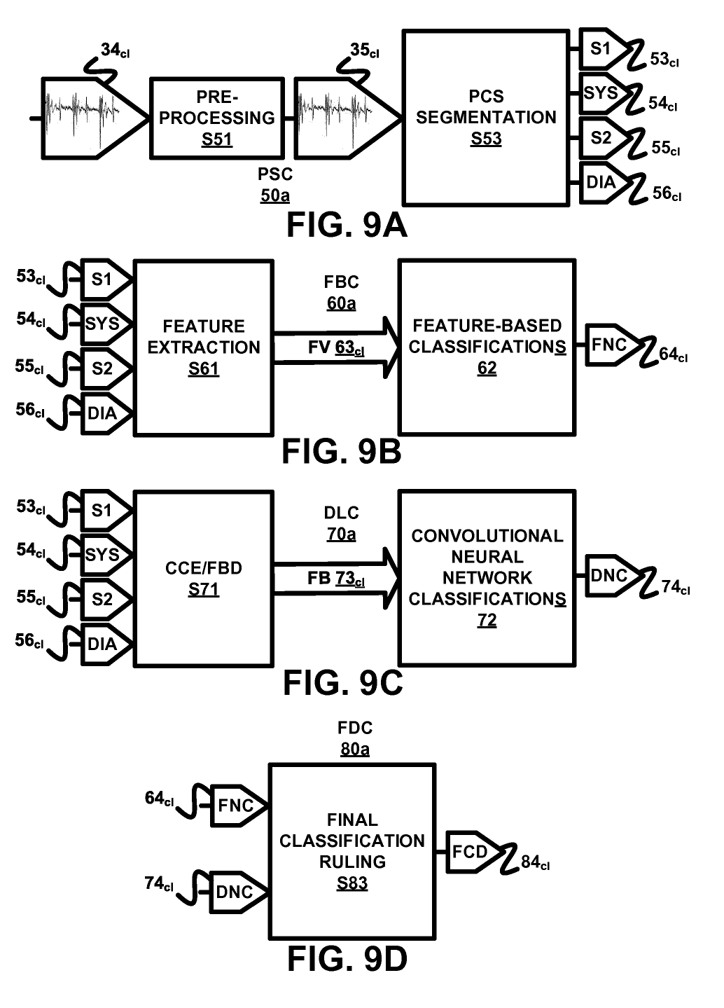

FIGS. 9A-9D illustrate an exemplary training of feature-based classifier 60a, deep learning classifier 70a and final decision coanalyzer 80a based on a training set of PCG signals 32ₙₘ delineated as clean PCG signals.

In practice, a training of feature-based classifier 60a involved 124 features were fed into the AdaBoost-abstain classifier to classify normal/abnormal heart sounds. Only fifty-nine (59) features were selected by the classifier after tuning parameters (e.g. number of iterations). Among the selected features, the top ten were the MFCC associated with S1, S2, and diastole states, SD of the kurtosis of the amplitudes during 51, and the mean and SD values of 51 and S2 intervals. AdaBoost—abstain provided an area under the receiver operating characteristic (AUC) of 0.91 on in-house test set.

A similar approach was used to classify noisy/clean heart sounds. Among 124 features as input to the AdaBoost-abstain classifier, sixty-nine (69) were selected by the classifier. The top ten features were related to the mean of the ratio of the mean absolute amplitude during systole to that during the S1 period, the mean absolute amplitude during diastole to that during the S2 period, mean value of RR intervals, the SD values of S2 and systole intervals, and the MFCC associated with S1, S2, and systole states. AdaBoost—abstain provided an AUC of 0.94 on the in-house test set.

In practice, a training of deep learning classifier 70a involved a tuning of hyperparameters of the CNN network using the in-house training set, resulting in the following configuration: batch size of 1024, learning rate of 0.0007, and 200 epochs. Early stoppage was applied when the loss function stop was decreasing. The CNN classier provided a AUC equal to 0.92 on the in-house test set for classification of normal/abnormal heart sound.

In practice, optimal levels for thresholds thr_SQI, thr_ABN, and thr_CNN were determined to be 0.7, 0.4, and 0.4, respectively. The results achieved on the in-house test set using the AdaBoost-abstain, the CNN, and classifier ensemble for classifying normal/abnormal heart sounds are shown in Table below. The best results using subset of blind test dataset was overall score of 0.885 (sensitivity and specificity equal to 0.96 and 0.809, respectively).

| Classifier | Sensitivity | Specificity | Overall Score |
|---|---|---|---|
| Adaboost-abstain | 0.82 | 0.88 | 0.85 |
| CNN | 0.93 | 0.86 | 0.89 |
| Classifier Ensemble | 0.96 | 0.80 | 0.89 |

Referring to FIGS. 1-9, those having ordinary skill in the art will appreciate the many benefits of the inventions of the present disclosure including, but not limited to, methods, systems and devices of the present disclosure providing a combination of a feature-based approach and a deep learning approach to facilitate an optimal accuracy for distinguishing between normal heart sounds and abnormal heart sounds.

Furthermore, it will be apparent that various information described as stored in the storage may be additionally or alternatively stored in the memory. In this respect, the memory may also be considered to constitute a “storage device” and the storage may be considered a “memory.” Various other arrangements will be apparent. Further, the memory and storage may both be considered to be “non-transitory machine-readable media.” As used herein, the term “non-transitory” will be understood to exclude transitory signals but to include all forms of storage, including both volatile and non-volatile memories.

While the device is shown as including one of each described component, the various components may be duplicated in various embodiments. For example, the processor may include multiple microprocessors that are configured to independently execute the methods described in the present disclosure or are configured to perform steps or subroutines of the methods described in the present disclosure such that the multiple processors cooperate to achieve the functionality described in the present disclosure. Further, where the device is implemented in a cloud computing system, the various hardware components may belong to separate physical systems. For example, the processor may include a first processor in a first server and a second processor in a second server.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A phonocardiogram (PCG) signal coanalyzer for distinguishing between normal heart sounds and abnormal heart sounds, the PCG signal coanalyzer comprising a processor and a memory configured to:
apply a feature-based classifier to a PCG signal to obtain a feature-based abnormality classification of the heart sounds represented by the PCG signal;
apply a deep learning classifier to the PCG signal to obtain a deep learning abnormality classification of the heart sounds represented by the PCG signal;
apply a final decision coanalyzer to the feature-based abnormality classification and the deep learning abnormality classification of the heart sounds represented by the PCG signal to determine a final abnormality classification decision of the PCG signal; and
report the final abnormality classification decision of the PCG signal.

2. The PCG signal coanalyzer of claim 1,
wherein the processor and memory are further configured to condition the PCG signal prior to applying the feature-based classifier and the deep learning classifier to the PCG signal; and
wherein a conditioning of the PCG signal includes of:
applying a spike filter to the PCG signal, and
segmenting the PCG signal into a plurality of heart sound states.

3. The PCG signal coanalyzer of claim 1, wherein an application of the feature-based classifier to the PCG signal includes:
extracting a feature vector from the PCG signal, the feature vector including of a time-domain feature and a frequency-domain feature; and
applying a AdaBoost-abstain classifier to the feature vector to determine the feature-based abnormality classification of the heart sounds represented by the PCG signal.

4. The PCG signal coanalyzer of claim 1, wherein an application of the deep learning classifier to the PCG signal includes:
extracting cardiac cycles from the PCG signal;
decomposing the cardiac cycles into frequency bands; and
applying a convolutional neural network to the frequency bands to determine the deep learning abnormality classification of the heart sounds represented by the PCG signal.

5. The PCG signal coanalyzer of claim 1, wherein an application of the final decision analyzer to the feature-based abnormality classification of the heart sounds represented by the PCG signal and the deep learning abnormality classification of the heart sounds represented by the PCG signal includes:
comparing abnormality threshold in accordance with a final decision rule to the feature-based abnormality classification of the heart sounds represented by the PCG signal and to the deep learning abnormality classification of the heart sounds represented by the PCG signal.

6. The PCG signal coanalyzer of claim 1, wherein the processor and memory are further configured to:
apply the feature-based classifier to a PCG signal to obtain a feature-based noisy classification of the heart sounds represented by the PCG signal; and
apply the final decision coanalyzer to the feature-based abnormality classification, the feature-based noisy classification and the deep learning abnormality classification of the heart sounds represented by the PCG signal to determine the final abnormality classification decision of the PCG signal.

7. The PCG signal coanalyzer of claim 1, wherein the processor and memory are further configured to:
apply the deep learning classifier to a PCG signal to obtain a deep learning noisy classification of the heart sounds represented by the PCG signal; and
apply the final decision coanalyzer to the feature-based abnormality classification, the deep learning abnormality classification and the deep learning noisy classification of the heart sounds represented by the PCG signal to determine the final abnormality classification decision of the PCG signal.

8. The PCG signal coanalyzer of claim 1, wherein the final abnormality classification decision of the PCG signal is one of:
a normal classification of the PCG signal; and
an abnormal classification of the PCG signal.

9. The PCG signal coanalyzer of claim 1, wherein the final abnormality classification decision of the PCG signal is one of:
a normal classification decision of the PCG signal;
an abnormal classification decision of the PCG signal; and
an unsure classification decision of the PCG signal.

10. The PCG signal coanalyzer of claim 1, wherein the PCG signal coanalyzer is in communication with a PCG signal recorder to receive the PCG signal.

11. A non-transitory machine-readable storage medium encoded with instructions for execution by a processor for distinguishing between normal and abnormal heart sounds, the non-transitory machine-readable storage medium comprising instructions to:
apply a feature-based classifier to a phonocardiogram signal to obtain feature-based abnormality classification of the heart sounds represented by the PCG signal;
apply a deep learning classifier to the PCG signal to obtain deep learning abnormality classification of the heart sounds represented by the PCG signal;
apply a final decision coanalyzer to the feature-based abnormality classification of the heart sounds represented by the PCG signal and the deep learning abnormality classification of the heart sounds represented by the PCG signal to determine a final abnormality classification decision of the PCG signal; and
report the final abnormality classification decision of the PCG signal.

12. The non-transitory machine-readable storage medium of claim 11,
wherein the non-transitory machine-readable storage medium further comprises instructions to condition the PCG signal prior to applying the feature-based classifier and the deep learning classifier to the PCG signal, and
wherein a conditioning of the PCG signal includes of:
applying a spike filter to the PCG signal, and
segmenting the PCG signal into a plurality of heart sound states.

13. The non-transitory machine-readable storage medium of claim 11, wherein an application of the feature-based classifier to the PCG signal includes:
extracting a feature vector from the PCG signal, the feature vector including of a time-domain feature and a frequency-domain feature; and
applying a AdaBoost-abstain classifier to the feature vector to determine the feature-based abnormality classification of the heart sounds represented by the PCG signal.

14. (canceled)

15. (canceled)

16. A phonocardiogram (PCG) signal coanalysis method for distinguishing between normal heart sounds and abnormal heart sounds, the PCG signal analysis method comprising:
applying a feature-based classifier to a PCG signal to obtain feature-based abnormality classification of the heart sounds represented by the PCG signal;
applying a deep learning classifier to the PCG signal to obtain deep learning abnormality classification of the heart sounds represented by the PCG signal;
applying a final decision coanalyzer to the feature-based abnormality classification of the heart sounds represented by the PCG signal and the deep learning abnormality classification of the heart sounds represented by the PCG signal to determine a final abnormality classification decision of the PCG signal; and
reporting the final abnormality classification decision of the PCG signal.

17. The PCG signal coanalysis method of claim 16, further comprising:
conditioning of the PCG signal prior to applying the feature-based classifier and the deep learning classifier to the PCG signal,
wherein the conditioning of PCG signal includes of:
applying a spike filter to the PCG signal, and
segmenting the PCG signal into a plurality of heart sound states.

18. (canceled)

19. (canceled)

20. (canceled)
